(19) United States
(12) Patent Application Publication
(10) Pub. No.: US 2021/0121694 A1
(43) Pub. Date: Apr. 29, 2021

(54) NON-INVASIVE METHOD FOR SUPPRESSING SPREADING DEPOLARIZATION IN HUMAN BRAINS

(71) Applicant: Carnegie Mellon University, Pittsburgh, PA (US)

(72) Inventors: Alireza Chaman Zar, Pittsburgh, PA (US); Pulkit Grover, Pittsburgh, PA (US); Shawn K. Kelly, Pittsburgh, PA (US); Maysam Chamanzar, Pittsburgh, PA (US); Shilpa George, Pittsburgh, PA (US); Anjali Menon, Pittsburgh, PA (US)

(21) Appl. No.: 17/082,251

(22) Filed: Oct. 28, 2020

Related U.S. Application Data
(60) Provisional application No. 62/973,839, filed on Oct. 28, 2019.

(51) Int. Cl. A61N 1/36; A61N 7/00; A61N 1/20

(52) U.S. Cl. CPC A61N 1/36025; A61N 7/00; A61N 1/36021; A61N 2007/0026; A61N 1/205

(57) ABSTRACT

Methods providing reliable and non-invasive ways to suppress spreading depolarization waves leading to cortical spreading depression are disclosed. The calcium conductance parameter of the 6 component Tuckwell model of spreading depolarization wave propagation is modified using Anderson Localization and other methods to disrupt the propagation of the waves, thus suppressing and eliminating spreading depolarization.

### RELATED APPLICATIONS

This application claims the benefit of U.S. Provisional Patent Application Ser. No. 62/973,839, filed Oct. 28, 2019, the contents of which are incorporated herein in their entirety.

### GOVERNMENT RIGHTS

This invention was made with government support under contract CNS1702694, issued by the National Science Foundation. The government has certain rights in this invention.

### BACKGROUND OF THE INVENTION

Migraines afflict 3.7 million Americans, and more than 1 billion individuals worldwide. Moreover, every year more than 3 million Acquired Brain Injuries (ABIs), which include traumatic brain injuries (TBIs), strokes, and hemorrhages, happen in the United States. These injuries are the leading cause of deaths and disabilities worldwide.

Cortical Spreading Depression (CSD) are waves of silencing of normal brain activity which propagate across the cerebral cortical surface. The slowly traveling wave (mm/min) is characterized by neuronal depolarization and redistribution of ions between the intracellular and extracellular space, that temporarily depresses electrical activity. This phenomenon is referred to as Spreading Depolarization (SD) and occurs in many neurological conditions, such as migraine with aura, ischemic stroke, traumatic brain injury and possibly epilepsy.

In the wake of the propagation of the SD wavefront, a complex dynamic is triggered. At first, neurons undergo a brief period of intense activity, exhibiting a firing rate 10-20 times higher than when at rest. This brief period of intense excitation is followed by a membrane hyperpolarization which silences the spiking activity for a variable period, after which the neurons slowly recover their spiking activity and eventually return to normal spiking frequency.

SD is characterized by relevant increases in both extracellular K⁺ and glutamate, as well as rises in intracellular Na⁺ and Ca⁺⁺. The two most accepted hypotheses suggest that propagation of the wave is due to diffusion of potassium or glutamate in the extracellular space.

SD has been shown to be responsible for worsening brain injuries, and can cause secondary brain damage after TBI, stroke, hemorrhages, etc. Commonly used techniques for SD suppression involve invasive and/or pharmacological methods. However, due to the side effects of surgery and chemical injections in the brain, and the time it takes to stop SD using these methods, new methods to suppress SD are needed. Therefore, finding a reliable, non-invasive way to suppress the SD is vital.

### SUMMARY OF THE INVENTION

SD is modelled herein using the standard Tuckwell model, the results of which are consistent with experimental findings about SD. Using this 2D model, SD was successfully suppressed by changing the calcium conductance term of the model. In three separate embodiments, the calcium conductance terms was changed in different ways, and, in all cases, the model shows that the SD was successfully suppressed.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1 are graphs showing the concentrations of the 6 components of the Tuckwell model at time steps t={1, 1000, 1500} of the simulation.

FIG. 2 is a graph showing membrane potential during SD wave propagation at time step t=1000.

FIG. 3 are graphs showing the extracellular K⁺ concentration (mM) at time steps t={0, 500, 800, 1200} for an embodiment of the invention using Anderson Localization.

FIG. 4 are graphs showing the extracellular concentrations (mM) of the 6 components of the Tuckwell model in one dimension (along the x-axis) at time step t=700.

FIG. 5 are graphs showing the extracellular concentrations (mM) of the 6 components of the Tuckwell model over time at x=y=90.

FIG. 6 shows a pattern for calcium conductance when an aspect of the invention providing localized stimulation is used.

### DETAILED DESCRIPTION

The Tuckwell mathematical models of SD wave propagation are considered in the form of reaction-diffusion systems in two space dimensions. SD consists of local “reaction” processes, such as a release of potassium and glutamate, pump activity and recovery of the tissue in a later stage, as well as diffusion of potassium and glutamate, which enables the propagation of SD.

The Tuckwell model of SD, with some modification, is used herein to evaluate the effectiveness of the invention in stopping SD. The model is based on extracellular (ECS) and intracellular (ICS) ion concentration changes during the SD propagation. In other words, the model is based on reaction-diffusion of different ions and transmitters. Reaction is referred to the ion exchange between ICS and ECS, and diffusion is referred to the ionic propagation in the ECS.

In the Tuckwell model, 6 components are considered: 4 ions (K⁺, Ca⁺⁺, Na⁺, Cl⁻) and two neurotransmitters, one inhibitory, Tl, such as GABA, and one excitatory, TE, which is mainly glutamate. Based on the ECS and ICS concentration of these components, the model comprises 6 coupled 2D parabolic partial differential equations (PDEs) as below:

$$\frac{\partial u}{\partial t} = {∇}^{2} u + F ( u ) \qquad ( 1 )$$

where:

u(x, y, t) is the vector of ECS concentrations with u₁, u₂, . . . , u₆ with the same order as K⁺, Ca⁺⁺, Na⁺, Cl⁻, TE and TI.

The initial condition for these concentrations are defined to put the neurons at rest condition. The initial values and constants used in this model, are:

u(x,y,0)=u₀(x,y) (2)

The boundary conditions were fixed at the initial concentrations. Because the propagation in these simulations starts at the middle of the 2D plane, this assumption seems to be reasonable.

Some assumptions in the SD model were made. First, the effect of action potentials is ignored for simplicity. This assumption is valid for SD modeling as there is some evidence to show that even in the absence of action potentials, SD can still propagate. For example, in case of TTX treatment, which suppresses action potential firing, SD can still propagate. Second, flows through other membranes are neglected to maintain a degree of simplicity. In addition, it is known that there is a relationship between the dynamics of calcium at the presynaptic region and the amount of transmitter release to the synaptic space, so a major component of calcium fluxes are included in this model.

For the ICS concentrations, there is another set of 4 unknown parameters (there is no TI or TE at ICS) and these parameters will be updated based on the difference between the current value of ECS concentrations and their resting equilibrium values, which are indicated by R. For intracellular concentrations of K⁺, Na⁺, Cl⁻, the following equation is used:

uᵢ^{int}(x,y,t)=uᵢ^{int.R}+α₁[uᵢ^{R}−uᵢ(x,y,t)],i=1,3,4 (3)

and for ICS Ca⁺ concentration:

u₂^{int}(x,y,t)=u₂^{int.R}+α₂[u₂^{R}−uᵢ(x,y,t)], (4)

In the model, the membrane potential can be computed using the Goldman formula:

$${V}_{M} = \frac{R T}{F} ln \frac{{K}^{o} + {p}_{N a} N {a}^{o} + {p}_{C l} C {l}^{i}}{{K}^{i} + {p}_{N a} N {a}^{i} + {p}_{C l} C {l}^{∘}} \qquad ( 5 )$$

In the same way, the Nernst potential for all of 4 ions can be computed as below:

$${V}_{K} = \frac{R T}{z F} ln \frac{{K}^{o}}{{K}^{i}} \qquad ( 6 )$$

where z is the ion charge.

In Eq. (1), the F(⋅) function is the flux term for each component which consists of two main parts, active pump current and passive fluxes through ion channels. For K⁺ this term can be calculated as below:
(7)

$${f}_{K} = {k}_{1} ( {V}_{M} - {V}_{K} ) [ \frac{{T}_{E}^{o} S ( {T}_{E}^{o} )}{{T}_{E}^{o} + {k}_{2}} + \frac{{k}_{3} {T}_{I}^{o} S ( {T}_{I}^{o} )}{{T}_{I}^{o} + {k}_{4}} ] + {f}_{K , p} + ( {k}_{5} - {P}_{K} ) \vert ( {K}^{o} \neq {K}^{o , R} )$$

where, Pₖ is the potassium active pump term and the last term is to make sure that this pump is only active when the concentrations are not at rest values.

In addition, f_{K,p} is the potassium passive flux term as below:

f_{K,p}=k₆(V_{M}−V_{M.R})(V_{M}−V_{K})S(V_{M}−V_{M.R}) (8)

where the S(⋅) function is a step function and V_{M.R} is the resting membrane potential.

There are two separate pump equations for sodium and potassium. These two exchange pump equations can be combined. Here, for potassium:

$${P}_{K} = \frac{{k}_{17} {K}^{o} {Na}^{i} S ( N {a}^{i} ) S ( {K}^{o} )}{{K}^{o} N {a}^{i} + {k}_{18} {K}^{o} + {k}_{19} N {a}^{i}} \qquad ( 9 )$$

and for sodium:

$${P}_{K} = \frac{{k}_{22} {K}^{o} N {a}^{i} S ( N {a}^{i} ) S ( {K}^{o} )}{{K}^{o} N {a}^{i} + {k}_{23} {K}^{o} + {k}_{24} N {a}^{i}} \qquad ( 10 )$$

For calcium, the F(⋅) function in Eq. (1), is defined as:

f_{Ca}=k₇(V_{M}−V_{Ca})g_{Ca}+(P_{Ca}−k₈)*1(Ca^{o}≠Ca^{o.R}) (11)

where the calcium conductance is defined as:

g_{Ca}=(1+tan h[k₃₁(V_{M}+V_{M}*)]−k₃₂)S(V_{M}−V_{M}^{T}) (12)

where, V_{M}^{T} is a cut-off potential and k₃₂ is defined as below to ensure a smooth rise of g_{Ca}, from zero:

k₃₂=1+tan h[k₃₁(V_{M}+V_{M}*)] (13)

In addition, calcium active pump can be defined as below:

$${P}_{C a} = \frac{{k}_{20} C {a}^{i} S ( C {a}^{i} )}{C {a}^{i} + {k}_{21}} \qquad ( 14 )$$

Similar equations exist for sodium and chloride ion fluxes:

$$\begin{matrix}{f}_{N a} = {k}_{9} ( {V}_{M} - {V}_{N a} ) [ \frac{{T}_{E}^{o} S ( {T}_{E}^{o} )}{{T}_{E}^{o} + {k}_{2}} + \frac{{k}_{10} {T}_{I}^{o} S ( {T}_{I}^{o} )}{{T}_{I}^{o} + {k}_{4}} ] + ( {P}_{N a} - {k}_{11} ) \vert ( N {a}^{o} \neq N {a}^{o , R} ) & ( 15 ) \\ {f}_{C l} = {k}_{12} ( {V}_{M} - {V}_{C l} ) [ \frac{{k}_{13} {T}_{E}^{o} S ( {T}_{E}^{o} )}{{T}_{E}^{o} + {k}_{2}} + \frac{{T}_{I}^{o} S ( {T}_{I}^{o} )}{{T}_{I}^{o} + {k}_{4}} ] + ( {P}_{C l} - {k}_{14} ) \vert ( C {l}^{o} \neq C {l}^{o , R} ) & ( 16 )\end{matrix}$$

where, the chloride active pump is as below:

$${P}_{Cl} = \frac{{k}_{25} C {l}^{i} S ( C {l}^{i} )}{C {l}^{i} + {k}_{26}} \qquad ( 17 )$$

Finally, based on the fact that rates of transmitter release are proportional to calcium flux, the transmitter's F(⋅) functions are given as:

f_{TE}−k₁₅(V_{M}−V_{Ca})g_{Ca}−P_{E} (18)

f_{TI}=k₁₆(Vₘ−V_{Ca})g_{Ca}−P_{I} (19)

where:

$$\begin{matrix}{P}_{E} = \frac{{k}_{27} {T}_{E}^{o} S ( {T}_{E}^{o} )}{{T}_{E}^{o} + {k}_{28}} & ( 20 ) \\ {P}_{I} = \frac{{k}_{29} {T}_{I}^{o} S ( {T}_{I}^{o} )}{{T}_{I}^{o} + {k}_{30}} & ( 21 )\end{matrix}$$

These pump terms work to return transmitters, such as GABA and glutamate, back to the glia cells. As previously stated, in this model, the effect of action potential firing and the consequent change in the ion concentrations are ignored and this can be translated into the fact that the effect of glutamate release from glia cells during SD have also been ignored.

For purposes of modeling embodiments of the invention, the SD propagation is instigated by means of a local elevation of ECS potassium concentration using KCL stimulation with a spatial-exponential profile as below:

$$\begin{matrix}{K}^{o} ( x , y , 0 ) = {K}^{o , R} + 17 exp [ - \{ {( \frac{x - 1}{0.05} )}^{2} + {( \frac{y - 1}{0.05} )}^{2} \} ] & ( 22 ) \\ C {l}^{o} ( x , y , 0 ) = C {l}^{o , R} + 17 exp [ - \{ {( \frac{x - 1}{0.05} )}^{2} + {( \frac{y - 1}{0.05} )}^{2} \} ] & ( 23 )\end{matrix}$$

To solve these coupled parabolic 2D partial differential equations, Euler's method can be used. The equations should first be discretized in this way:

$$\frac{1}{Δ t} ( {U}_{m}^{n} - {U}_{m}^{n - 1} ) = \frac{D}{2 Δ {x}^{2}} [ {U}_{m + 1}^{n - 1} - 2 {U}_{m}^{n - 1} ] + F ( \frac{3}{2} {U}_{m}^{n - 1} - \frac{1}{2} {U}_{m}^{n - 2} ) \qquad ( 24 )$$

where, D is the scaled diffusion coefficient of the corresponding component

$$( \times 1 {0}^{2} \frac{{cm}^{2}}{sec} ) .$$

In this solution, two previous time indices, n−1 and n−2, are used to make the updates of the parameters smoother. In this simulation, the spatial unit step is equal to 5.2 mm and the temporal unit step is equal to about 26 sec. The dimension of the 2D plane in this simulation is a 2×2 spatial unit and the total duration of simulation is 10 temporal units.

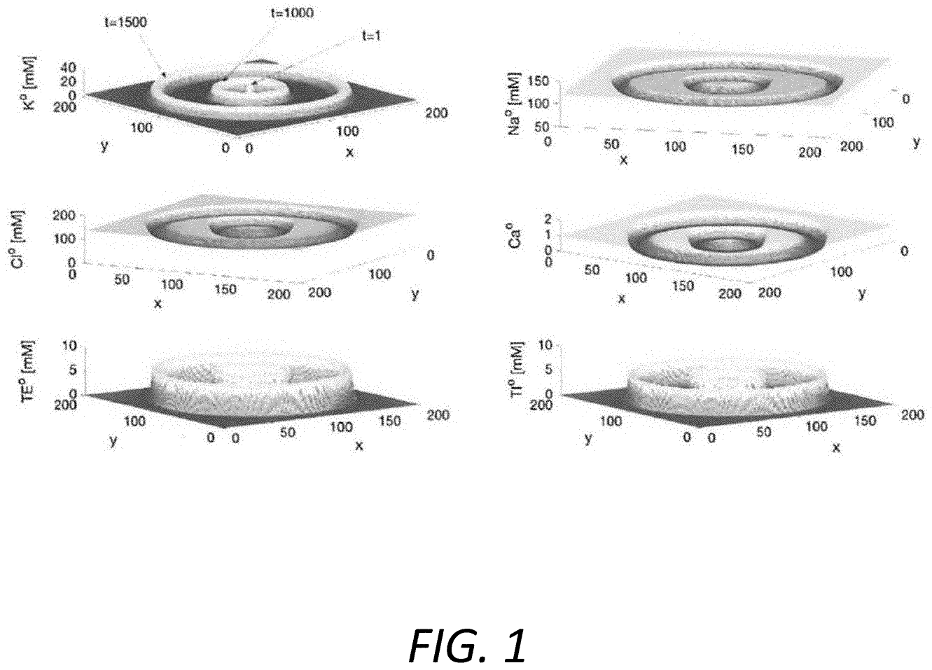

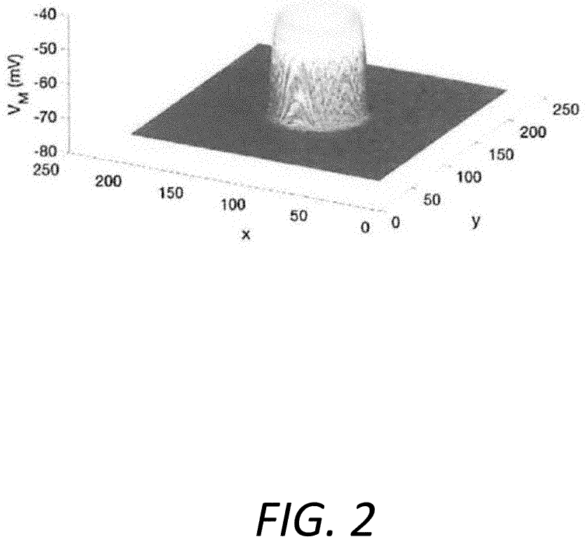

As shown in FIG. 1, the changes in the ECS concentrations of all 6 components of the Tuckwell model are shown at three different time points, which are obtained by means of numerical solution of the above equations. These results are consistent with the experimental findings, but there are still some unknowns. In particular, the physiological mechanism of increasing ECS potassium concentration, other than action potential, during SD propagation still remains a question. FIG. 2 is an illustration of membrane potential (V_{M}) in 2D. As can be see, during the SD propagation, there is a slow increase (because of the slow speed of propagation of SD) in the membrane potential, and this increase is around the required threshold for action potential firing, but, surprisingly, there is no firing. One possible explanation for this is that because the increase in the amplitude of membrane potential is gradual, the cell does not fire.

Once the model of SD propagation has been implemented, the next task at hand is SD suppression.

In a first embodiment of the invention, the calcium conductance was randomized by applying Anderson Localization. Anderson localization is a general concept that may be applied to any wave system. It entails the randomization of the medium through which the wave is travelling and is used in this invention to disrupt the SD wave. In case of SD, this is randomization of the cortical medium. Parameters related to firing rates and threshold potentials were randomized to achieve Anderson localization.

In the Tuckwell model, the calcium conductance term g_{Ca}, depends on the membrane voltage, and, as such, can be randomized using Anderson Localization. The calcium conductance term g_{Ca}, in turn controls the flux terms of Ca⁺⁺, Glutamate and GABA in the 6 component Tuckwell model. In this embodiment of the invention, the constant k₃₁ is replaced by a Gaussian random process.

k₃₁→ [symbol] (k₃₁,k₃₁²) (25)

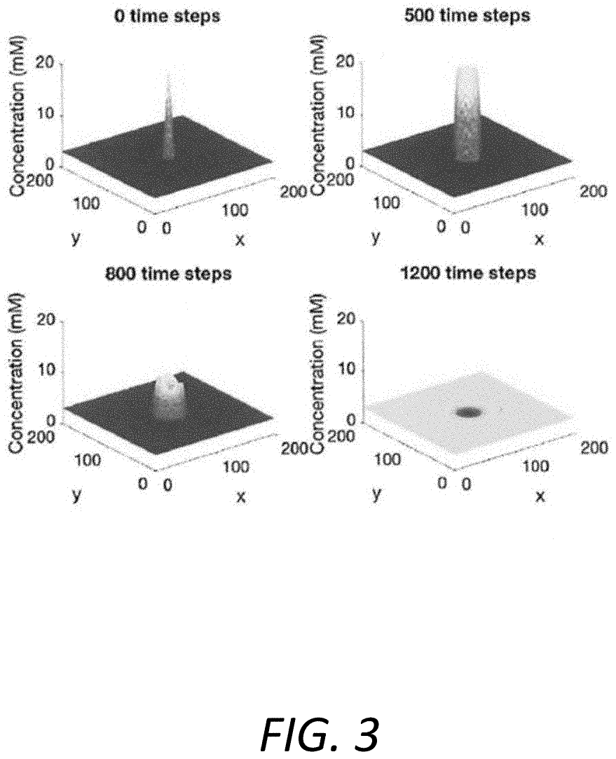

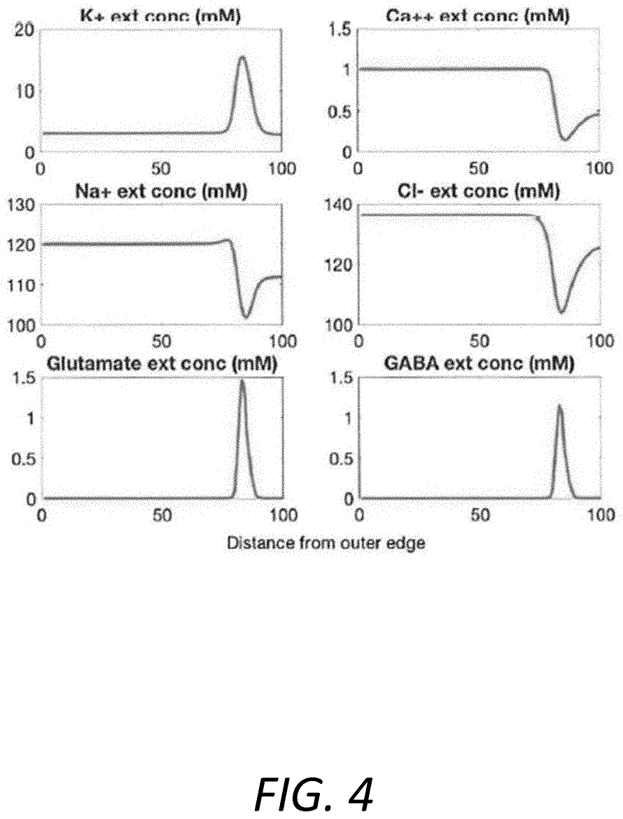

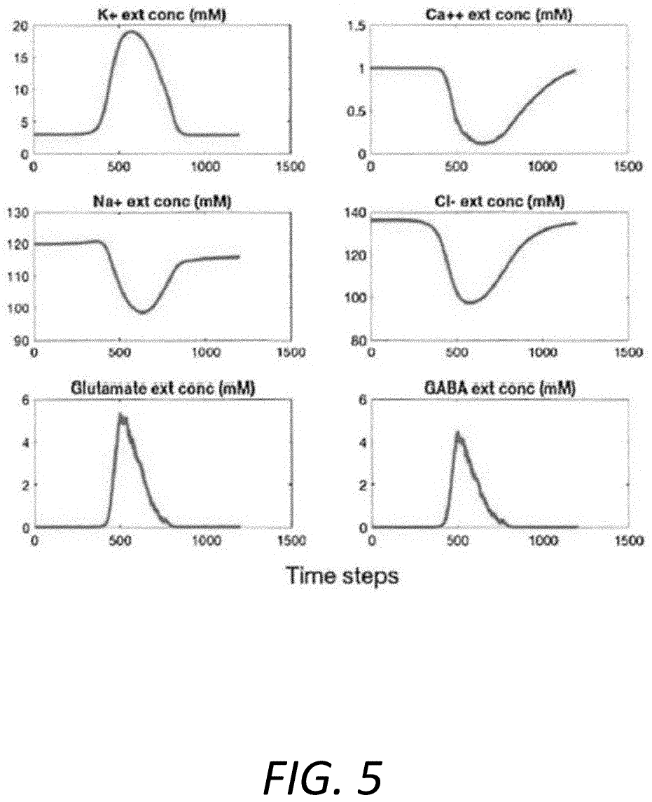

Randomizing the k₃₁ parameter in accordance with the Gaussian function results in SD suppression. The result of randomizing calcium conductance on extracellular K⁺ concentrations is shown in FIGS. 3-5. The randomization was introduced at t=500 time steps. FIG. 3 shows that the SD suppression after randomization is introduced is clear. FIG. 4 and FIG. 5 show the extracellular concentrations of all 6 components of the Tuckwell model at a specific instant of time (FIG. 4) and at a specific location (x=y=90, FIG. 5). This embodiment of the invention may be implemented for use on an actual brain using a transcranial random noise stimulation (TRNS) process to randomize the calcium conductance in the intracellular space by randomizing both frequency and amplitude of the electrical signal injected into the brain.

In a second embodiment of the invention, the calcium conductance term may be scaled in accordance with a constant.

k₃₁→ck₃₁ (26)

where c is a constant that will either increase of decrease the k₃₁ parameter.

In real life scenarios, this embodiment may be implemented using a transcranial direct current stimulation (TDCS) process to scale the calcium conductance in the intracellular space. In a separate aspect of this embodiment, the value of c may be varied around a mean by randomly changing the amplitude of the TDCS signal.

In yet a third embodiment of the invention, the calcium conductance term may be changed in accordance with a spatial sinusoidal wave function in which may be implemented using a transcranial alternating current stimulation (TACS) process to change the calcium conductance in the intracellular space in accordance with a deterministic wave.

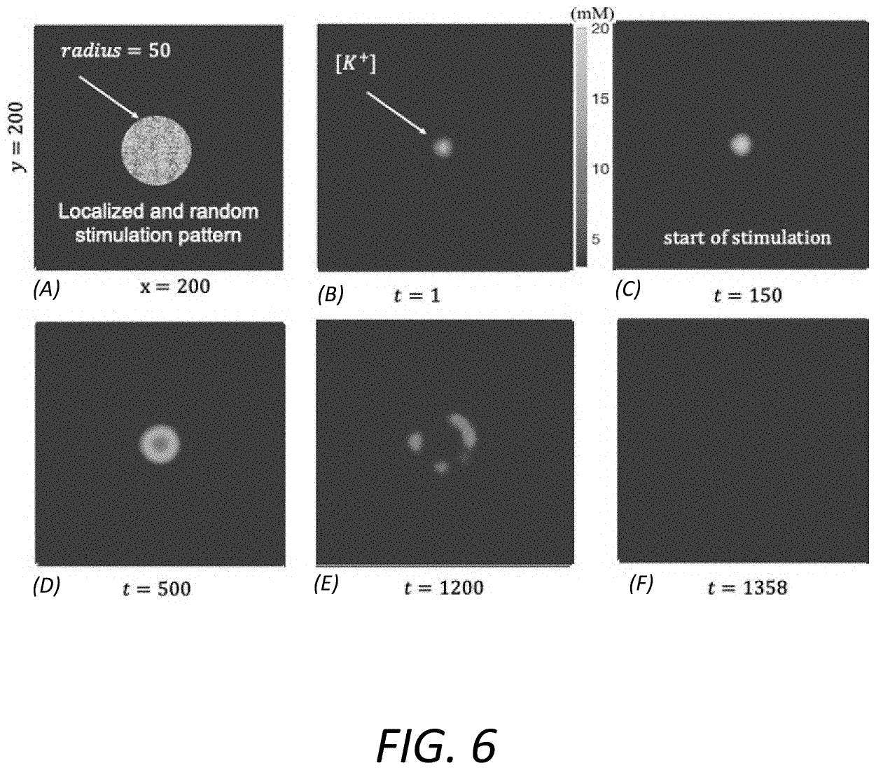

In an aspect of the invention, all three of the described embodiments may be localized to a particular area of the brain experiencing SD to provide localized change in the calcium conductance as opposed to a global, brain-wide change. Localized randomization of calcium conductance is equally effective to rapidly and completely suppress and inhibit the SD in the cortical space. FIG. 6 shows this result. FIG. 6(A) shows the spatial pattern of stimulation is shown for k₃₁ (Eq. (12) describes how g_{Ca} changes as a function of k₃₁). This pattern is a localized randomization of k₃₁ inside a disk with a radius of only r=50 (in a 200×200 Tuckwell model), where the center of the disk is aligned with the origin of an instigated SD (x₀, y₀) in this model:

$${k}_{31} = \{ \begin{matrix}𝒩 ( c {k}_{31} , {k}_{31}^{2} ) , c > 1 , & {( x - {x}_{0} )}^{2} + {( y - {y}_{0} )}^{2} \leq {r}^{2} and t \geq {t}_{0} \\ {k}_{31} , & {( x - {x}_{0} )}^{2} + {( y - {y}_{0} )}^{2} \geq {r}^{2} or t \leq {t}_{0}\end{matrix} \qquad ( 27 )$$

This stimulation pattern is applied at t₀=150, as shown in FIG. 6(C), and continues until the full suppression of SD at t=1358, shown in FIG. 6(F). Such localized stimulation patterns may be achieved in practice using current stimulation methods such as temporal interference (TI).

In another aspect of the embodiments of the invention, a phased array of ultrasound transducers may be used to provide neuromodulation.

Neuromodulation using ultrasound has also been seen to stimulate Na⁺ and Ca⁺⁺ channel activity leading to an increased firing of neurons. These may have a role to play in neurotransmitter-based ion channels. Ultrasound has the advantage of being non-invasive, although incurring the cost of poor spatial resolution. This kind of neuromodulation is known to affect membrane permeability which, in turn, would have an influence on conductance. In the Tuckwell model, only calcium conductance is assumed to play a significant role towards SD propagation and conductance associated with K⁺ and Na⁺ have been ignored. Using ultrasonic neuromodulation, it is possible to randomize calcium conductance to achieve the simulated result shown in FIG. 3.

Cortical spreading depression caused by SD has been implicated in migraine and as a headache trigger and, in the case of an acutely injured brain, the depolarization waves cause secondary neuronal damage. Understanding the mechanism of SD is paramount to the objective of suppressing these waves. It is clear that a large number of neural, glial, synaptic, metabolic and neurochemical variables are involved in some way with the formation and passage of a SD wave.

This invention has introduced several methods of suppressing the SD waves by modifying concentrations of the ions and neurotransmitters important to the sustenance of SD. In some embodiments, the invention described herein includes methods that can vary neural parameters, particularly, but not limited to, calcium conductance across space and time, to suppress SD waves.

1. A method of suppressing spreading depolarization in a brain comprising:
modifying the calcium concentration in intracellular spaces of the brain.

2. The method of claim 1 wherein the calcium concentration is modified by introducing an electrical signal into the brain.

3. The method of claim 1 wherein the calcium concentration is modified by introducing ultrasonic waves into the brain.

4. The method of claim 1 wherein the spreading depolarization is modeled in accordance with a Tuckwell model and further wherein modifying the calcium concentration comprises modifying the k₃₁ parameter of the model.

5. The method of claim 1 wherein the electrical signal causes the calcium concentration to be randomized.

6. The method of claim 5 wherein the calcium concentration is randomized in accordance with a standard Gaussian distribution.

7. The method of claim 5 wherein the calcium concentration is randomized by introducing an electrical signal into the brain using a transcranial random noise stimulation process.

8. The method of claim 1 wherein the electrical signal causes the calcium concentration to be scaled by a constant.

9. The method of claim 8 when the electrical signal causes the calcium concentration to be randomized about a mean of the constant.

10. The method of claim 8 wherein the calcium concentration is scaled by introducing the electrical signal into the brain using a transcranial direct current stimulation process.

11. The method of claim 1 wherein the electrical signal causes the calcium concentration to be modulated in accordance with a deterministic wave function.

12. The method of claim 11 wherein the calcium concentration is modulated by introducing the electrical signal into the brain using a transcranial alternating current stimulation process.

13. The method of claim 1 wherein the modification of the calcium concentration occurs in a localized portion of the brain experiencing the spreading depolarization.
